(19) United States
(12) Patent Application Publication
(10) Pub. No.: US 2010/0152765 A1
(43) Pub. Date: Jun. 17, 2010

(54) BODY LUMEN FILTERS WITH STRUCTURES TO REDUCE PARTICULATES AND METHODS FOR FILTERING A BODY LUMEN

(75) Inventor: Charlene A. Haley, LaBelle, FL (US)

Correspondence Address: WORKMAN NYDEGGER, 1000 EAGLE GATE TOWER,, 60 EAST SOUTH TEMPLE, SALT LAKE CITY, UT, 84111, US

(73) Assignee: Abbott Laboratories, Abbott Park, IL (US)

(21) Appl. No.: 12/537,097

(22) Filed: Aug. 6, 2009

Related U.S. Application Data
(60) Provisional application No. 61/138,455, filed on Dec. 17, 2008.

(51) Int. Cl. A61F 2/01

(52) U.S. Cl. 606/200

(57) ABSTRACT

As described herein, a body lumen filter is provided that includes a body configured to move between a pre-deployed state and a deployed state, a filtering structure operatively associated with the body to filter particulates, and a separating structure operatively associated with the body. The separating structure can be configured to break up at least one of the particulates.

### CROSS-REFERENCE TO RELATED APPLICATIONS

This U.S. Patent Application claims the benefit of and priority to U.S. Provisional Patent Application having Ser. No. 61/138,455, filed on Dec. 17, 2008, the disclosure of which is incorporated herein by reference in its entirety.

### BACKGROUND

1. Field of the Invention

The present disclosure relates generally to medical devices. More specifically, the present disclosure generally relates to body lumen filters with structure to reduce particulates and methods for filtering a body lumen.

2. Background and Relevant Art

Surgical procedures, including both invasive as well as minimally-invasive procedures, save countless lives each year. However, the instrument and processes used during such procedures sometimes create additional challenges. For example, many minimally invasive procedures are performed using highly specialized surgical tools that are introduced to the procedure site by way of the patient's vasculature. In such a minimally invasive procedure, a catheter is introduced into the vasculature by way of small incision. The catheter is then advanced into proximity with the procedure site. Thereafter, the surgical tools are advanced to the procedure site through the catheter. With the surgical tools thus at the procedure site, the surgical tools are then manipulated from the outside of the body. Accordingly, a surgical procedure may be performed with only a small incision. While such an approach may reduce the invasiveness of performing a surgical procedure, this approach may cause additional challenges.

In particular, as the catheter and/or surgical devices are advanced through the vasculature, their passage may cause arterial plaques, clots, or other debris commonly referred to as thrombi to become dislodged and move with the blood as it circulates through the vasculature. Additionally, patients with reduced mobility may develop thrombi that may also become dislodged. As the emboli move downstream, they may encounter plaque or other obstructions within the bloodstream to form new clots or obstructions in the bloodstream. Such obstructions can result in partial or complete blockage of vessels supplying blood and oxygen to critical organs, such as the heart, lungs and brain.

Accordingly, filter devices have been developed to capture the emboli at safe locations. Vena cava filters are devices that are implanted in the inferior vena cava, providing a mechanical barrier to undesirable particulates. The filters may be used to filter peripheral venous blood clots and other particulates, which if remaining in the blood stream can migrate in the pulmonary artery or one of its branches and cause harm.

While such filters may capture the emboli at a safe location, the functionality of the filter may be reduced as more emboli are captured, which may reduce the flow of blood through the filter. Further, if conventional filters move or become tilted within the vasculature, the functionality of the filter may be compromised as the embolus-trapping area may be reduced.

### BRIEF SUMMARY

As described herein, a body lumen filter is provided that includes a body configured to move between a pre-deployed state and a deployed state, a filtering structure operatively associated with the body to filter and/or lyse particulates, and a separating structure operatively associated with the body to fractionate particulates encountering the filtering structure. This separating structure can be configured to break up, divide, reduce, or otherwise decrease the size of at least one of the particulates.

In at least one example, the separating structure may be an active-type separating structure. In particular, an active body lumen filter may include a generally spherically-shaped outer member configured to move between a pre-deployed state and a deployed state. The outer member includes a plurality of filtering gaps defined therein. The active body lumen filter also includes an inner member located at least partially within the outer member when the outer member is in the deployed state. The inner member is configured to move in response to a flow of body fluid to break up, divide, reduce, or otherwise decrease the size of at least one type of particulates carried by a fluid flow.

In other examples, the body lumen filter may include a stationary separating structure. In particular, the body lumen filter can include a body being configured to move between a pre-deployed state and an deployed state. The body includes a filtering structure including at least first filtering gaps defined therein. The body lumen filter can also include a plurality of individual stationary separating features operatively associated with the body. The plurality of individual stationary separating features can be configured to break up particulates carried by a fluid flow.

These and other features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 illustrates schematic view of a body lumen filter according to one example;

FIG. 2A illustrates a body lumen filter according to one example;

FIG. 2B illustrates a body lumen filter in a pre-deployed state and being introduced to a body lumen by a deployment device according to one example;

FIG. 2C illustrates a body lumen filter in an deployed state and deployed in the body lumen according to one example;

FIGS. 2D-2E illustrate a retrieval process for a body lumen filter according to one example;

FIG. 3A illustrates a body lumen filter deployed in a body lumen according to one example;

FIG. 3B is a front plan view of a body lumen filter according to the example shown in FIG. 3A;

FIG. 3C is a front plan view of a body lumen filter according to another example;

FIG. 3D is a perspective view of a body lumen filter according to a further example;

FIGS. 3E-3F illustrate a retrieval process for a body lumen filter according to one example;

FIG. 4A illustrates a body lumen filter deployed in a body lumen according to one example;

FIG. 4B is a front plan view of the body lumen filter according to the example shown in FIG. 4A;

FIG. 4C illustrates a retrieval process for a body lumen filter according to one example;

FIG. 5A illustrates a body lumen filter according to one example;

FIG. 5B illustrates a body lumen filter in a pre-deployed state and being introduced to a body lumen by a deployment device according to one example;

FIG. 5C illustrates a body lumen filter in an deployed state and deployed in the body lumen according to one example;

FIG. 5D illustrates a retrieval process for a body lumen filter according to on example;

FIG. 6A illustrates a body lumen filter deployed in a body lumen according to one example; and

FIG. 6B illustrates a retrieval process for a body lumen filter according to one example.

### DETAILED DESCRIPTION

Apparatuses and methods are provided herein for filtering a flow of body fluid, such as a blood flow passing through a body lumen. By way of example only, a body lumen may include a blood vessel. Filtering may be performed by body lumen filters. For instance, embodiments of body lumen filters (e.g. including vena cava and/or other lumen filters), are described. Components of body lumen filters also are described. These components may include anchors and/or other components. In particular, the apparatuses and methods provided herein include body lumen filters having both a filtering structure as well as a separating structure operatively associated therewith. According to some examples, the separating structure is a stationary separating structure while in other examples the separating structure is active. Several examples of body lumen filters having both stationary separating structures as well as active separating structures will be described in more detail below. The separating structures discussed below are configured to reduce the size of at least some of the particulates, such as emboli, that the separating structures engage.

For example, the separating structures may fractionate the particulates until at least a portion of the remaining emboli are below a selected size, such as a size that has been determined to be less threatening to the patient. For ease of reference, a blood vessel and blood will be discussed as the type of body lumen and body fluid filtered by the body lumen filter. Further, the filtering structure and separating structure may be discussed separately. It will be appreciated that other lumens and fluids may be filtered and that filtering and separating functionality may be performed by the same structure. Additionally, the filtering openings or gaps shown in the drawings are shown schematically to illustrate the functionality of the body lumen filters and are not necessarily drawn to scale.

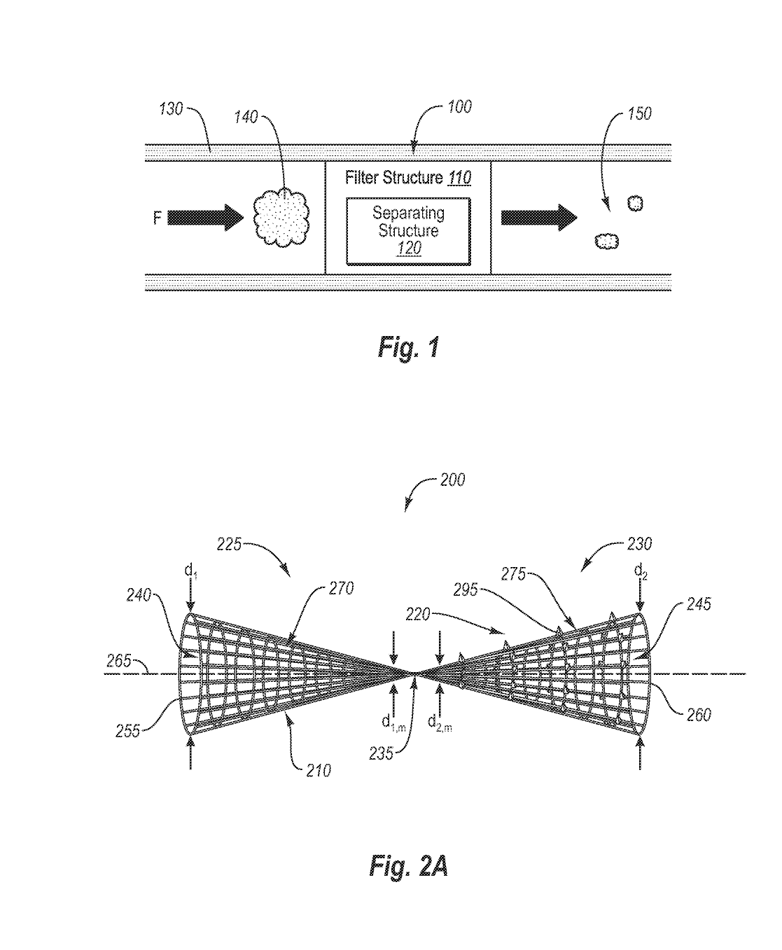

FIG. 1 schematically illustrates a body lumen filter 100 for filtering a body fluid, such as a blood flow. The body lumen filter 100 includes a filtering structure 110 as well as a separating structure 120. The blood flows through a body lumen 130 and through the body lumen filter 100 when deployed within the body lumen 130. The blood flowing through the body lumen 130 flows in the direction indicated by the arrow F. The body lumen filter 100 may capture and/or lyse particulates, such as an embolus 140 (or emboli) that are carried by the blood flow. For ease of reference, a single embolus 140 will be described as being filtered and fractionated. It will be appreciated that any number of emboli with of any number of sizes and having any number of characteristics may be filtered and/or lysed by body lumen filters as described in more detail below.

In particular, the body lumen filter 100 includes a filtering structure 110 and a separating structure 120. The blood flow may engage the filtering structure 110 and/or the separating structure 120. For instance, the filtering structure 110 includes a number of filtering openings defined therein. The filtering openings in the filtering structure 110 allow blood to flow through the filtering structure 110 while capturing and/or lysing embolus 140 or other particulates that are larger than the filtering openings thereby preventing emboli of particular sizes from passing downstream of the body lumen filter 100.

In addition to capturing and/or lysing the embolus 140 with the filtering structure 110, the body lumen filter 100 includes separating structure 120 that engages the embolus 140 to fractionate (break up, divide, reduce, lyse, or otherwise decrease the size of) the embolus 140 into smaller particles or emboli. The separating structure 120 may be configured to reduce the size of the embolus 140 to a size that may be less threatening when allowed to pass downstream of the body lumen filter 100.

The size of the openings in the filtering structure 110 described above may be of a size that allows less threatening particles, including fractionated emboli particles 150 to pass therethrough. The filtering structure 110 and the separating structure 120 have been illustrated and described separately for ease of reference only. It will be appreciated that the filtering structure 110 may perform separating functions and the separating structure 120 may perform filtering functions.

In several examples, the separating structure may include stationary separating structures. Other examples may include active separating structures or separating structures that move to fractionate the emboli. For ease of reference, stationary separating structures and active separating structures will be described separately. It will be appreciated that body lumen filters may either be active separating structures, stationary separating structures, or some combination of the two.

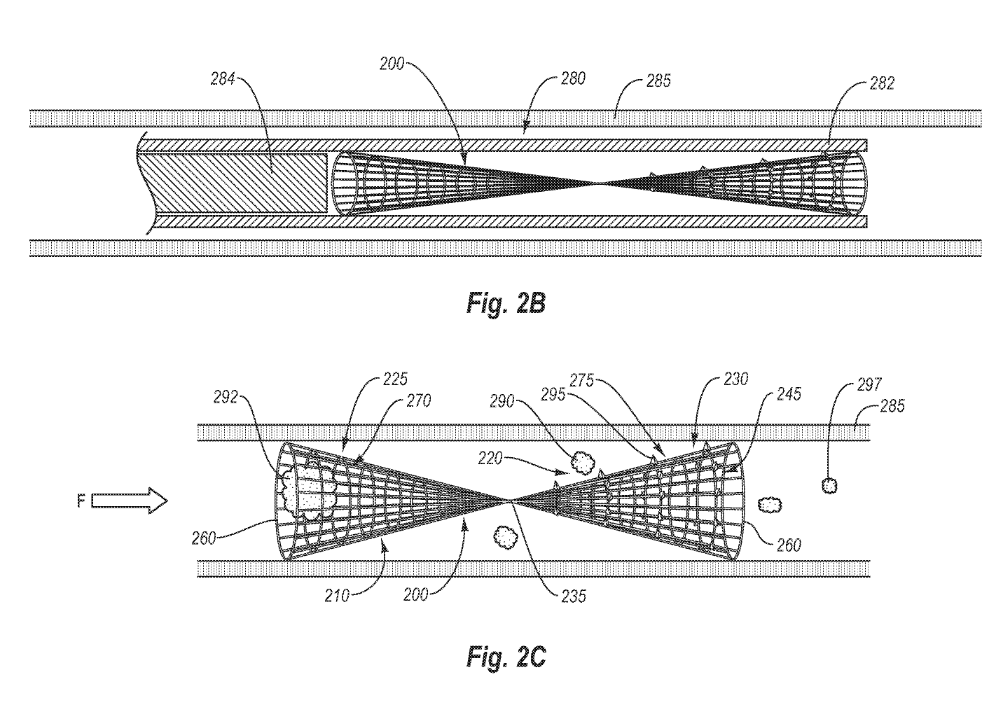

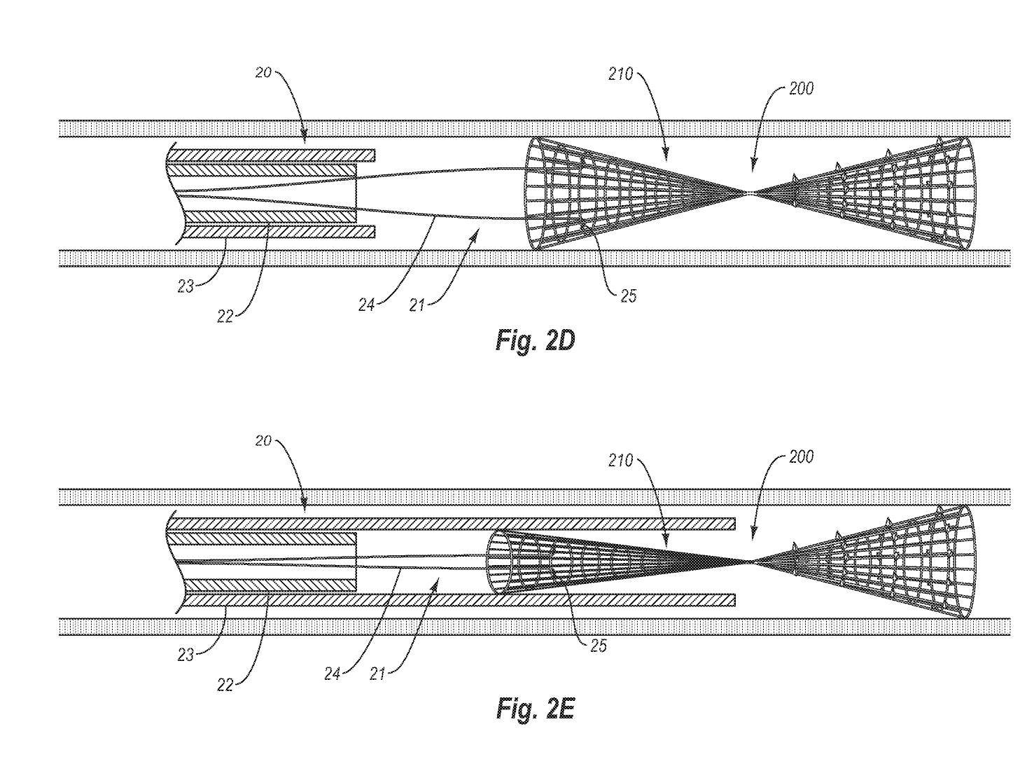

FIG. 2A illustrates a body lumen filter 200 having filtering structure 210 as well as stationary separating structure 220. The stationary separating structure 220 may include a plurality of generally pointed (i.e. cuspate or aciculate) portions at various locations on the filtering structure 210. In the illustrated example, the filtering structure generally includes a first portion 225 and a second portion 230 that are coupled together by an intermediate portion 235. For ease of reference, surfaces of the body lumen filter 200 proximate the intermediate portion 235 will be referred to as interior surfaces, while surfaces opposing the interior surfaces will be referred to as exterior surfaces.

In the illustrated example, the first portion 225 has a first opening 240 defined therein that extends completely through the first portion 225. The second portion 230 can include a second opening 245 defined therein. In at least one example, the first opening 240 has a generally conical shape such that the first opening 240 has a first dimension d1 near a first exterior portion 255 and a first intermediate dimension d1,m near the intermediate portion 235. The first opening 240 can transition smoothly from the first dimension d1 to the first intermediate dimension d1,m. In other examples, the first opening 240 can transition in a stepwise fashion or in any other manner.

The second opening 245 can also have a generally conical shape such that the second opening 245 transitions smoothly from a second dimension d2 near a second exterior portion 260 toward a second intermediate dimension d2,m near the intermediate portion 235. In other examples, the second opening 245 can transition in a step-wise fashion or in any other fashion. The first intermediate dimension d1,m and the second intermediate d2,m may be the same or they may be different. Further, the intermediate portion 235 may have any dimensions. In addition, in other examples, the intermediate portion 235 may have an opening defined therein in communication with each of the first opening 240 and the second opening 245.

In the example illustrated, the body lumen filter 200 is disposed along a central axis 265 such that the first portion 225, the second portion 230, and the intermediate portion 235 are centered relative to the central axis 265. Similarly, the first opening 240 and the second opening 245 may also be centered relative to the central axis 265. In other examples, any or all of the first portion 225, the second portion 230, the intermediate portion 235, the first opening 240, the second opening 245 or combinations thereof may not be centered relative to the central axis 265.

Regardless of the orientation of these parts, in at least one example the first portion 225, as well as the second portion 230, includes first and second filtering openings 270, 275 defined therein. The first and second filtering openings 270, 275 are configured to allow body fluids to flow therethrough while filtering blood particulates such as emboli while deployed.

FIG. 2B illustrates the body lumen filter 200 located within a deployment device 280 used to deploy the filter 200 into a body lumen 285. The deployment device 280 may include a housing 282 and a delivery mechanism 284 that may be actuated from a proximally located handle (not shown). In particular, each of the first portion 225, the second portion 230, and the intermediate portion 235 (all shown best in FIG. 2A) can be formed of annular elements, helical elements, crossbars, connectors, junctions, braids, and other like features. The separating structure 220, best illustrated in FIGS. 2A and 2C, may be formed integrally with the elements discussed above or may be formed afterward, such as through a deposition process and/or a joining process. Further, the body lumen filter 200 may be formed of a resilient material. Such a configuration may allow the device to move between the deployed or unstressed state illustrated in FIG. 2A and the stressed or pre-deployed state illustrated in FIG. 2B, in which the body lumen filter 200 is located within the housing 282.

To deploy the body lumen filter 200, the deployment device 280 is moved near a desired location within a body lumen 285 by using a catheter or other well-known techniques. Once the deployment device 280 is near the desired location, the delivery mechanism 284, such as a plunger or other moveable member disposed within the housing 282, may be advanced distally relative to the housing 282, thereby moving the body lumen filter 200 from the housing 282. As the body lumen filter 200 is advanced from the housing 282, the body lumen filter 200 moves towards the deployed and/or unstressed state. For instance, when the body lumen filter 200 is formed from a shape memory material, such that, moving the delivery mechanism 284 distally, moving the housing 282 proximally, or a combination of such movements, releases the body lumen filter 200 from within the housing 282 to transition to the deployed, unstressed state illustrated in FIG. 2C. A resilient body lumen filter 200 is illustrated. Embodiments of the body lumen filter body can include a material made from any of a variety of known suitable materials, such as a shape memory material (SMM). For example, the SMM can be shaped in a manner that allows for restriction to induce a substantially reduced, generally linear orientation while within the housing 282, but can automatically return to the memory shape of the body lumen filter 200 once extended from the deployment device 280. SMMs have a shape memory effect in which they can be made to remember a particular shape. Once a shape has been remembered, the SMM may be bent out of shape or deformed and then returned to its original shape by unloading from strain and/or heating. Typically, SMMs can be shape memory alloys (SMA) comprised of metal alloys, or shape memory plastics (SMP) comprised of polymers. The materials can also be referred to as being superelastic.

Usually, an SMA can have any non-characteristic initial shape that can then be configured into a memory shape by heating the SMA and conforming the SMA into the desired memory shape. After the SMA is cooled, the desired memory shape can be retained. This allows for the SMA to be bent, straightened, compacted, and placed into various contortions by the application of requisite forces; however, after the forces are released, the SMA can be capable of returning to the memory shape. The main types of SMAs are as follows: copper-zinc-aluminum; copper-aluminum-nickel; nickel-titanium (NiTi) alloys known as nitinol; and cobalt-chromium-nickel alloys nickel-titanium platinum; nickel-titanium palladium or cobalt-chromium-nickel-molybdenum alloys known as elgiloy alloys. The temperatures at which the SMA changes its crystallographic structure are characteristic of the alloy, and can be tuned by varying the elemental ratios or by the conditions of manufacture.

For example, the primary material of an body lumen filter 200 can be of a NiTi alloy that forms superelastic nitinol. In the present case, nitinol materials can be trained to remember a certain shape, straightened in a shaft, catheter, or other tube, and then released from the catheter or tube to return to its trained shape. Also, additional materials can be added to the nitinol depending on the desired characteristic. The alloy may be utilized having linear elastic properties or non-linear elastic properties.

A SMP is a shape-shifting plastic that can be fashioned into an endoprosthesis in accordance with the present invention. Also, it can be beneficial to include at least one layer of an SMA and at least one layer of an SMP to form a multilayered body; however, any appropriate combination of materials can be used to form a multilayered body lumen filter 200. When an SMP encounters a temperature above the lowest melting point of the individual polymers, the blend makes a transition to a rubbery state. The elastic modulus can change more than two orders of magnitude across the transition temperature (Ttr). As such, an SMP can formed into a desired shape of an endoprosthesis by heating it above the Ttr, fixing the SMP into the new shape, and cooling the material below Ttr. The SMP can then be arranged into a temporary shape by force, and then resume the memory shape once the force has been applied. Examples of SMPs include, but are not limited to, biodegradable polymers, such as oligo(ε-caprolactone)diol, oligo(ρ-dioxanone)diol, and non-biodegradable polymers such as, polynorborene, polyisoprene, styrene butadiene, polyurethane-based materials, vinyl acetate-polyester-based compounds, and others yet to be determined. As such, any SMP can be used in accordance with the present invention.

A body lumen filter body having at least one layer made of an SMM or suitable superelastic material and other suitable layers can be compressed or restrained in its delivery configuration within a delivery device using a sheath or similar restraint, and then deployed to its desired configuration at a deployment site by removal of the restraint. A body lumen filter body made of a thermally-sensitive material can be deployed by exposure of the endoprosthesis to a sufficient temperature to facilitate expansion. It will be appreciated that the body lumen filter 200 may be mechanically deployed, such as by a balloon or other expanding device.

FIG. 2C illustrates the body lumen filter 200 deployed within the blood vessel 285. Blood particulates of various sizes, such as emboli 290, 292 may be carried in the blood flow in the direction indicated by arrow F. In at least one example, the first filtering openings 270 may be larger than the second filtering openings 275. It will be appreciated that other configurations are possible, including configurations in which the first filtering openings 270 are the same size as the second filtering openings 275 or sizes in which the second filtering openings 275 are larger than the first filtering openings 270. It will be also understood, that the openings 270 and 275 may be differently sized along the length or diameter of first portion 225 or second portion 230.

Continuing with the example illustrated in FIG. 2C, the emboli may include differentially sized emboli, as represented by a smaller embolus 290 and a larger embolus 292. The smaller embolus 290 may be able to pass through the first filtering openings 270 while the larger embolus 292 may not pass through the filtering openings 270. In at least one example, the first filtering openings 270 may allow particulates that are smaller than a largest less threatening size to pass, as represented by the smaller embolus 290.

The smaller embolus 290 then travels toward the second portion 230 of the body lumen filter 200. As illustrated in FIG. 2C, the separating structure 220 may be located on the interior surface of the second portion 225. As a result, the smaller embolus 290 moves into engagement and/or contact with the separating structure 220. While the separating structure 220 is illustrated on the interior surface of the second portion 225, it will be appreciated that the separating structure may be located on any of the other surfaces, such as the interior and exterior surfaces of the first portion 225.

In the example illustrated, the separating structure 220 includes a plurality of individual members 295 that fractionate embolus contacting the individual member 295. Each of the individual members 295 may include one or more surfaces configured to break up or divide particulates, such as the smaller embolus 290. The individual members 295 may include one or more features that engage the smaller embolus 290, such as a tip and/or bladed or sharpened edges.

For instance, FIG. 2C illustrates a smaller embolus 290 engaging an individual member 295. As previously introduced, when the smaller embolus 290 engages one or more of the individual members 295, the blood flow causes the smaller embolus 290 to be fractionated by the engagement with the features of the individual member 295. In at least one example, the individual member 295 may include at least one cutting edge 296 and/or a cuspate end 297. In the illustrated example, the cuspate end 297 has a single point. It will be appreciated that the individual member 295 may also include a non-cuspate end and/or a cuspate end having more than one point.

As previously introduced, the flow of fluid may carry the emboli through the body lumen 285. The fluid flow that carries the smaller embolus 290 through the body lumen 285 (FIGS. 2B-2C) may carry the smaller embolus 290 through the body lumen 285 into engagement with the individual member 295. For instance, the pointed tip of the cuspate end 297 may pierce the smaller embolus 290, thereby engaging the smaller embolus 290 at that location. The fluid flowing on the smaller embolus 290 may then exert a force on the smaller embolus 290. The penetration of the cuspate end 297 into the embolus may then cause the smaller embolus 290 to fractionate in response to the force exerted by the fluid flow and/or other forces. If the individual member 295 includes a cutting edge 296, the cutting edge 296 may further facilitate fractionation of the smaller embolus 290 by providing a relatively sharp edge that may cut through the portion of the smaller embolus 290 that has been pierced by the individual member 295. Accordingly, the individual members 295 may be configured to fractionate smaller emboli, such as finer emboli 297.

The finer emboli 297 may be broken up to a size that allows it to pass through the second filtering openings 275. The second filtering openings 275 may be of such a size that particulates that are able to pass therethrough may be of a less threatening size. As a result, the body lumen filter 200 is configured to filter particulates of a threatening size as well as to reduce the size of at least some of the particulates to a less threatening size.

FIGS. 2D and 2E illustrate a process for removing the body lumen filter 200 according to one example. As illustrated in FIG. 2D, a retrieval device 20 may be positioned in proximity to the body lumen filter 200. The retrieval device 20 can include an engagement device 21, an inner housing 22, and an outer housing 23. In at least one example, the engagement device 21 can include expandable arms 24 having engagement feature 25, such as hooks. In other embodiments, the retrieval device 20 may include other features to facilitate retrieval of the body lumen filter 200. The expandable arms 24 and engagement features 25 can be positioned within the inner housing 22 as the retrieval device 20 is moved into proximity with the body lumen filter 200. Further, the inner housing 22 and/or the engagement device 21 can be positioned within the outer housing 23 as the retrieval device 20 is moved into proximity with the body lumen filter 200.

Thereafter, the engagement device 21 and/or the inner housing 22 can be moved distally out of the outer housing 23. As the engagement device 21 is urged out of the inner housing 22 and/or the outer housing 23, the expandable arms 24 can move radially outwardly. As the expandable arms 24 thus expand radially, the engagement features 25 can come into engagement with the body lumen filter 200, such as into engagement with the filtering structure 210. The expandable arms 24 can then be moved radially inward, such as by moving the inner housing 22 distally relative to the expandable arms 24.

As the expandable arms 24 move radially inward, engagement between the engagement features 25 and the body lumen filter 200 can reduce the radial dimension of the associated portion of the body lumen filter 200. Thereafter, the engagement device 21 and the body lumen filter 200 can be drawn into the outer housing 23, as illustrated in FIG. 2E. Once the body lumen filter 200 is positioned with in the outer housing 23, the retrieval device 20 and body lumen filter 200 can be withdrawn.

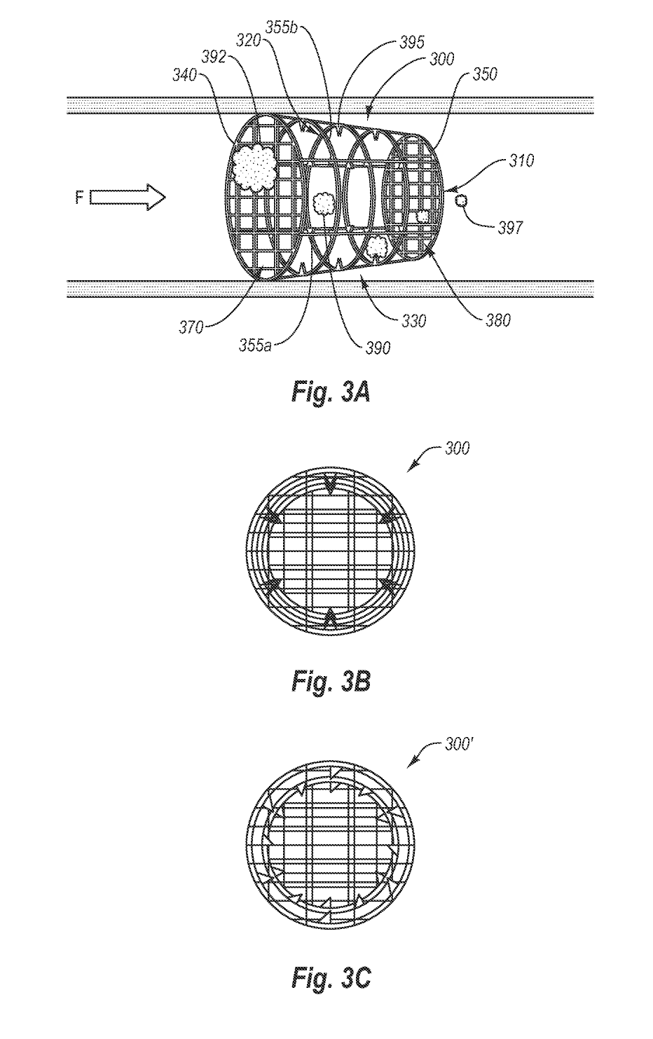

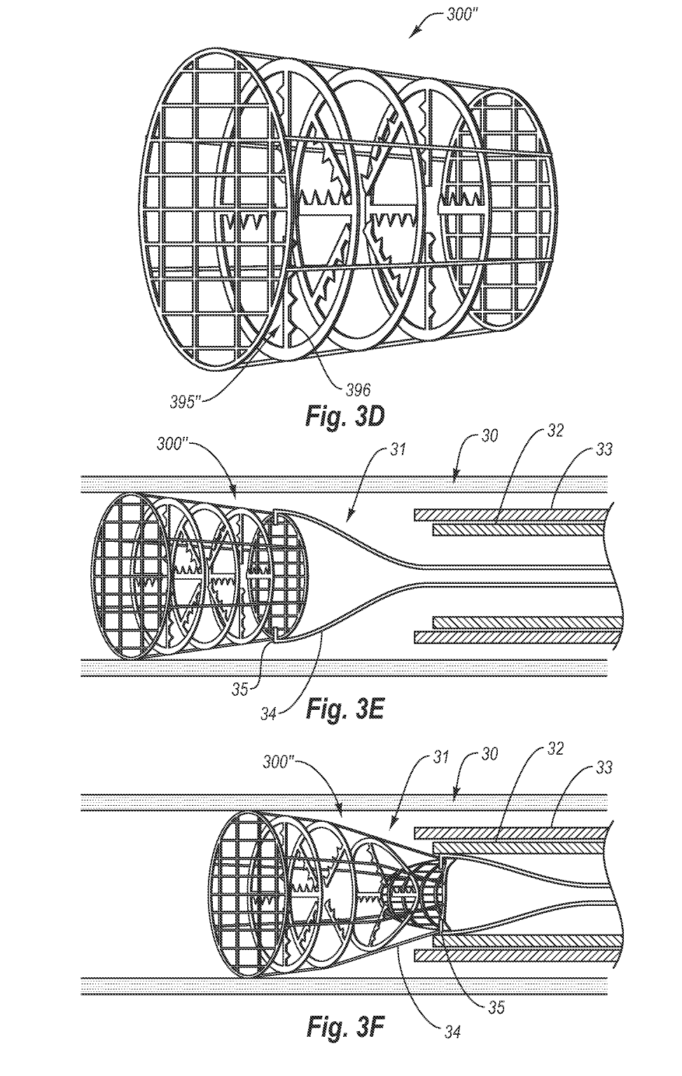

FIGS. 3A-3D illustrate another example of body lumen filter 300 that can both filter and fractionate emboli from flowing blood. The body lumen filter 300, as illustrated, includes filtering structure 310 as well as separating structure 320. The filtering structure 310 may include a main body 330 having an inlet 340 as well as an outlet 350. In the illustrated example, the inlet 340 has a larger cross-sectional area than the cross-sectional area of the outlet 350. The main body 330 may include vertical struts 355A as well as horizontal struts 355B. The vertical struts 355A and the horizontal struts 355B may combine to form a perimeter portion that transition between the inlet 340 and the outlet 350, in a stepwise fashion, or in any other manner.

In at least one example, the inlet 340 includes first filtering openings 370 defined therein while the outlet 350 includes second filtering openings 380 defined therein. The first filtering openings 370 may be larger than the second filtering openings 380. The first filtering openings 370 may allow a small embolus 390 to pass while filtering and/or lysing large embolus 392, which represent particulates both of which are above a largest less threatening size.

The small embolus 390 may come into contact with individual members 395 that are operatively associated with one or more of the vertical struts 355A and/or the horizontal struts 355B. The individual members 395 may break the smaller embolus 390 into finer emboli 397. Further, the finer emboli 397 may be of sufficient size to be less threatening and allowed to pass through the second filtering openings 380. In other examples, the body lumen filter 300 may include individual members 395 on only one of its ends, such as on the outlet 350.

FIG. 3B illustrates the body lumen filter 300 in which the inlet 340 is open and viewed from the inlet. The individual members 395 may be associated with any combination of the vertical struts 355A and the horizontal struts 355B. In the illustrated example, individual members 395 are associated with each of the vertical struts 355A and the horizontal struts 355B. The individual member 395 illustrated may extend radially toward the center of the body lumen filter. It will be appreciated that other configurations are possible in which the individual members are operatively associated with other portions of the vertical struts 355A and/or horizontal struts 355B. Further, as illustrated in FIG. 3B, some of the individual members 395 may overlap or otherwise be configured.

In other examples, such as that illustrated in FIG. 3C, a body lumen filter 300′ may include individual members 395′ that are angled away from a radial direction as the individual members 395 extend away from the vertical struts 355A and/or the horizontal struts 355B. For instance, the individual members 395′ may be oriented away from a longitudinal axis of the body lumen filter 300′.

FIG. 3D illustrates individual members 395″ that extend from vertical struts 355A. The individual members 395″ may be multi-cuspate individual members that may include a plurality of cuspate portions 398. It will be appreciated that the configurations of individual members described herein as well as other configurations may be applied to any of the body lumen filters described herein in any combination.

FIGS. 3E and 3F illustrate a process for removing the body lumen filter 300″ according to one example. As illustrated in FIG. 3E, a retrieval device 30 may be positioned in proximity to the body lumen filter 300″ from an opposing direction as the deployment shown in FIG. 3D. The retrieval device 30 can include an engagement device 31, an inner housing 32, and an outer housing 33. In at least one example, the engagement device 31 can include expandable arms 34 having engagement features 35, such as hooks. In other embodiments, the retrieval device 30 may include other features to facilitate retrieval of the body lumen filter 300″. The expandable arms 34 and engagement features 35 can be positioned within the inner housing 32 as the retrieval device 30 is moved into proximity with the body lumen filter 300″. Further, the inner housing 32 and/or the engagement device 31 can be positioned within the outer housing 33 as the retrieval device 30 is moved into proximity with the body lumen filter 300″.

Thereafter, the engagement device 31 and/or the inner housing 32 can be moved distally out of the outer housing 33. As the engagement device 31 is urged out of the inner housing 32 and/or the outer housing 33, the expandable arms 34 can move radially outwardly. As the expandable arms 34 thus expand radially, the engagement features 35 can come into engagement with the body lumen filter 300″. The expandable arms 34 can then be moved radially inward, such as by moving the inner housing 32 distally relative to the expandable arms 34.

As the expandable arms 34 move radially inward, engagement between the engagement features 35 and the body lumen filter 300″ can reduce the radial dimension of the associated portion of the body lumen filter 300″. Thereafter, the engagement device 31 and the body lumen filter 300″ can be drawn into the outer housing 33, as illustrated in FIG. 3E. Once the body lumen filter 300″ is positioned with in the outer housing 33, the retrieval device 30 and body lumen filter 300″ can be withdrawn.

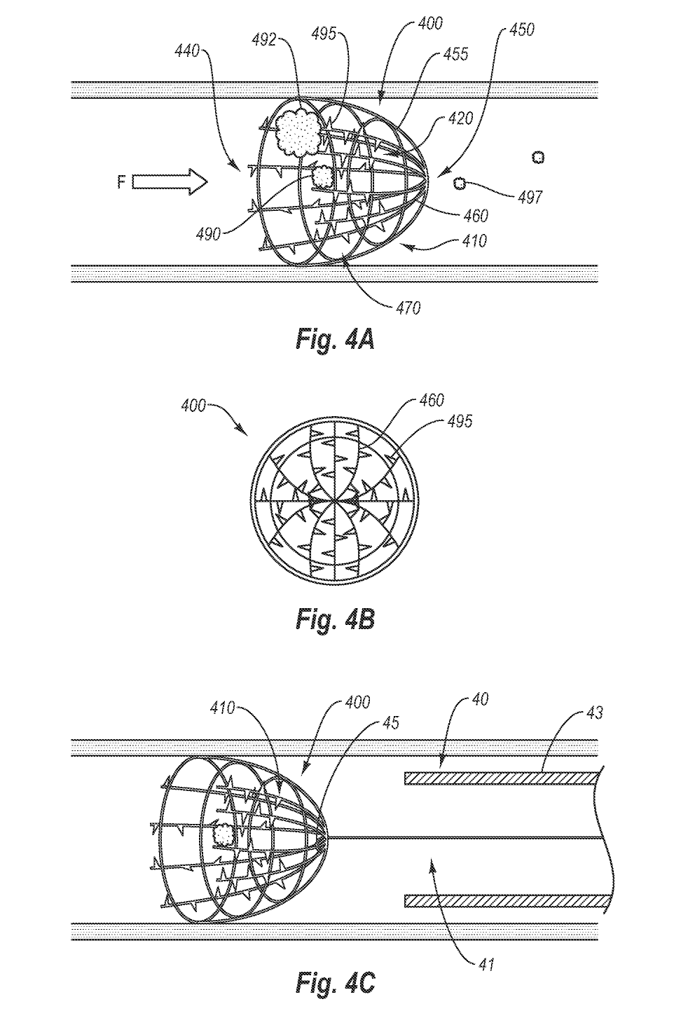

FIG. 4A illustrates another example of a body lumen filter 400 having a filtering structure 410 and a stationary separating structure 420. In the example illustrated in FIG. 4A, the generally basket-shaped body lumen filter 400 includes an inlet 440 as well as an outlet 450. The inlet 440 may be open while walls 455 extend from, proximate to, or distal to the inlet 440 that may define the outlet 450. The body lumen filter 400 may also include ribs 460 that may extend into the opening between the inlet 440 and the outlet 450 of the body lumen filter 400. In other examples, the stationary separating structure 420 may be formed on the walls 455 and/or the ribs 460 may be omitted. Individual members 495 may be located on the walls 455 and/or the ribs 460. The individual members 495 may be configured in a similar manner to other individual members described herein. For instance, the individual members 495 can break emboli 490, 492 into finer emboli 497 that may pass through filtering openings 470 defined in the walls 455 and the outlet 450. Accordingly, body lumen filters have been discussed that include stationary separating structures.

FIG. 4B illustrates an end view of the body lumen filter 400. As illustrated in FIG. 4B, the ribs 460 may be distributed in such a manner as to locate a large number of individual members 495 in the space occupied by the deployed body lumen filter 400. Such a configuration may provide a relatively large chance that particulates, such as emboli, may engage one or more of the individual members 495. It will be appreciated that any of the individual members 495 may be configured in any manner. For example, individual members 495 may be configured in a similar manner or configuration to the individual members discussed above.

FIG. 4C illustrate a process for removing the body lumen filter 400 according to one example. As illustrated in FIG. 4C, a retrieval device 40 may be positioned in proximity to the body lumen filter 400 from an opposing direction as the deployment shown in FIG. 4A. The retrieval device 40 can include an engagement device 41 and an outer housing 43. In at least one example, the engagement device 41 includes an engagement feature 45, such as a hook. In other embodiments, the retrieval device 40 may include other features to facilitate retrieval of the body lumen filter 400. The engagement feature 45 can be positioned within the outer housing 43 as the retrieval device 40 is moved into proximity with the body lumen filter 400.

Thereafter, the engagement device 41 can be moved distally out of the outer housing 43. As the engagement device 41 is urged out of the outer housing 43, the engagement feature 45 can come into engagement with the body lumen filter 400, such as into engagement with the filtering structure 410. Thereafter, the engagement device 41 and the body lumen filter 400 can be drawn into the outer housing 43. Once the body lumen filter 400 is positioned within the outer housing 43, the retrieval device 40 and body lumen filter 400 can be withdrawn.

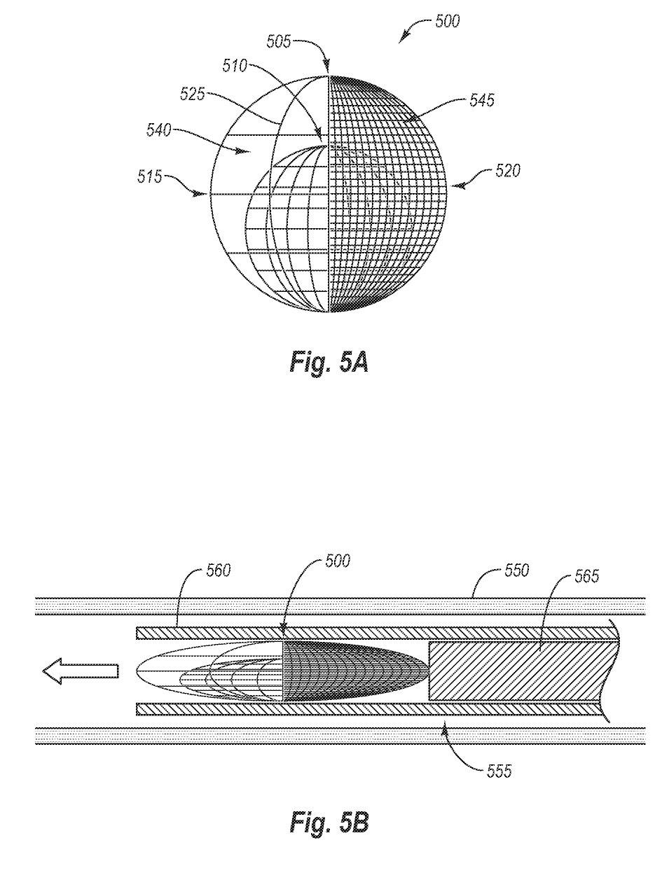

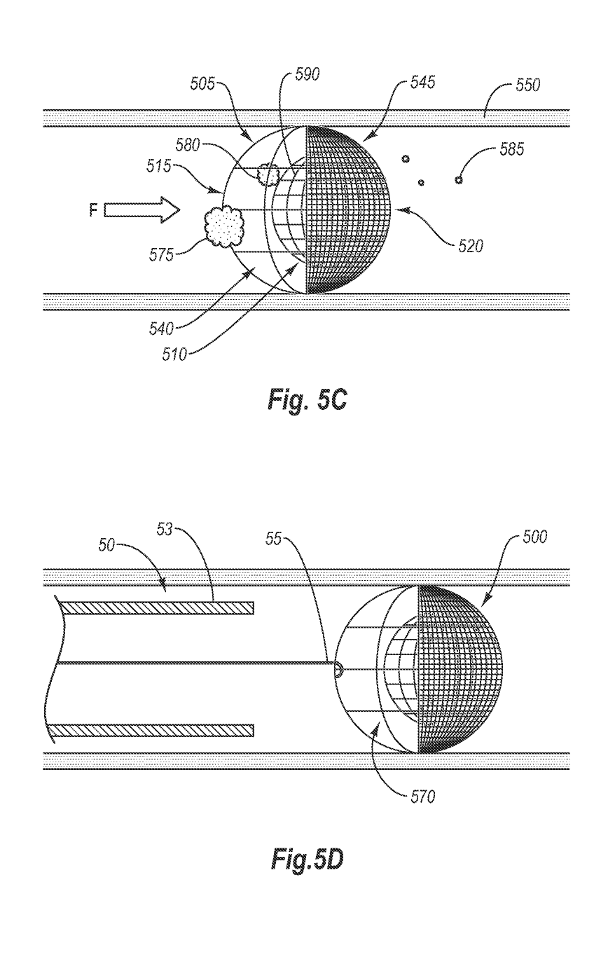

FIG. 5A illustrates a partial cross-sectional view of a body lumen filter 500 that includes an outer member 505 and an inner member 510 in an unstressed or deployed state. The outer member 505 and the inner member 510 may cooperate to provide active separating of particulates, such as emboli. Each of the outer member 505 and the inner member 510 are illustrated as having a generally spherical shape. It will be understood, however, that various other configurations are possible that allow the inner member 510 to be movably disposed within the outer member 505 and the outer member 505 to contact and/or engage with a vessel wall.

In the illustrated example, the outer member 505 may be divided into a first hemispherical portion 515 and a second hemispherical portion 520. Each of the first hemispherical portion 515 and the second hemispherical portion 520 include supports 525. Further, the first hemispherical portion 515 and the second hemispherical portion 520 each may include a filter screen associated therewith. The outer member 505 may include both a plurality of first filtering openings 540 and/or a plurality of second filter openings 545 defined therein. The first filtering openings 540 may be generally larger, approximately the same size, or smaller than the second filtering openings 545.

As illustrated in FIG. 5B, the body lumen filter 500 is configured to be deployed into a body lumen 550. In particular, FIG. 5B illustrates the body lumen filter 500 located within a deployment device 555. The deployment device 555 may include a housing 560 and/or a delivery mechanism 565 that may actuated from a proximally located handle (not shown). Each of the outer member 505 and the inner member 510 can be formed of annular elements, helical elements, crossbars, connectors, junctions, braids, other like features, or combinations thereof.

Further, the body lumen filter 500 may be formed of a resilient material. Such a configuration may allow the device to move between the deployed or unstressed state illustrated in FIG. 5A and the stressed or pre-deployed state illustrated in FIG. 5B, in which the body lumen filter 500 is located within the housing 560.

The body lumen filter 500 may be deployed by advancing the delivery mechanism 565 relative to the housing 560 to thereby move the body lumen filter 500 distally of the housing 560. As the body lumen filter 500 is advanced, the body lumen filter 500 may move toward the deployed or unstressed state and into engagement with the body lumen 550 as illustrated in FIG. 5C. In another example, the deployment device 555 may be advanced to the desired location, the delivery mechanism 565 may be advanced distally to abut the body lumen filter 500, the housing 560 may be retracted to deploy the body lumen filter 500, or combinations thereof. As previously introduced, the outer member 505 and/or the inner member 510 may be generally hemispherical in shape.

Such a configuration may increase the likelihood of proper functioning of the body lumen filter 500 regardless of how the body lumen filter 500 is deployed or whether it moves after deployment. In particular, the hemispherical shape of the outer member 505 may provide consistent engagement of the body lumen filter 500 and the body lumen 550 when the body lumen filter 500 is twisted or otherwise changes orientation. Consistent engagement may provide for increased reliability of the body lumen filter 500.

As further illustrated in FIG. 5C, the first filtering gaps 540 may filter and/or lyse particulates larger than a given size, such as a large embolus 575 while allowing smaller particulates, such as small embolus 580, to enter the outer member 505. As the small embolus 580 enters the outer member 505, the small embolus 580 may be engaged and/or lysed by the inner member 510.

In the example illustrated, the inner member 510 may also include supports 590 that support a membrane (not shown). The membrane may be semi-permeable so as to allow body fluid, such as blood, to pass therethrough. Further, the membrane may be sufficiently stiff to help break the small embolus 580 carried with the blood flow into finer emboli 585.

In particular, in at least one example, the body fluid flows in the direction indicated by the arrow F. Further, the flow of body fluid may fluctuate as blood is pulsed through the body lumen as a heart beats. The fluctuation of the flow may cause the inner member 510 to move into and out of engagement with the outer member 505. Further, the fluctuation and/or other forces may cause the inner member 510 to rotate and/or move relative to the outer member 505.

The engagement between the inner member 510 and the outer member 505 may break the small embolus 580 into finer emboli 585. If the finer emboli 585 are sufficiently small as to be less threatening, the finer emboli 585 may pass through the second filtering gaps 545 in the second hemispherical portion 520. The breaking of the small embolus 580 into finer emboli 585 may be described as a separating of the small embolus 580.

Examples have been described in which substantially the entire outer portion 505 is covered with a filtering structure. In other examples, a portion of the outer member 505 may be uncovered by a portion of either the first and/or the second filtering structure 530, 535. For example, a substantial portion of the first hemispherical portion 520 may be substantially open, except for the supports 590 to allow large and small emboli 575, 580 to enter the body lumen filter 500.

In such an example, the supports 590 may act to retain the inner member 510 while allowing the inner member 510 to engage the second hemispherical portion 520 to thereby reduce the size of particulates, such as emboli 575, 580. In other examples, the inner member includes different components.

The outer member 505 and the inner member 510 may be formed of wires and/or mesh. Further, the wires and/or mesh may be formed of a resilient material, such as shape memory materials including nitinol. For example, the inner member 510 may be entirely formed first, after which the outer member 505 may be partially formed. In particular, in one example a wire frame may be formed of members aligned in one direction. Thereafter, the inner member 510 may be collapsed and inserted between adjacent members in the wire frame and then allowed to expand. Thereafter, additional wires and/or mesh may be secured to the outer member 505 to provide the filtering gaps described above.

FIG. 5D illustrates a process for removing the body lumen filter 500 according to one example. As illustrated in FIG. 5D, a retrieval device 50 may be positioned in proximity to the body lumen filter 500 from an opposing direction as the deployment shown in FIG. 5A. The retrieval device 50 can include an outer housing 53 and an engagement feature 55, such as a hook. In other embodiments, the retrieval device 50 may include other features to facilitate retrieval of the body lumen filter 500. The engagement feature 55 can be positioned within the outer housing 53 as the retrieval device 50 is moved into proximity with the body lumen filter 500.

Thereafter, the engagement feature 55 can be moved distally out of the outer housing 53. As the engagement feature 55 is urged out of the outer housing 53, the engagement feature 55 can come into engagement with the body lumen filter 500 as shown. Thereafter, the engagement feature 55 and the body lumen filter 500 can be drawn into the outer housing 53. Once the body lumen filter 500 is positioned with in the outer housing 53, the retrieval device 50 and body lumen filter 500 can be withdrawn.

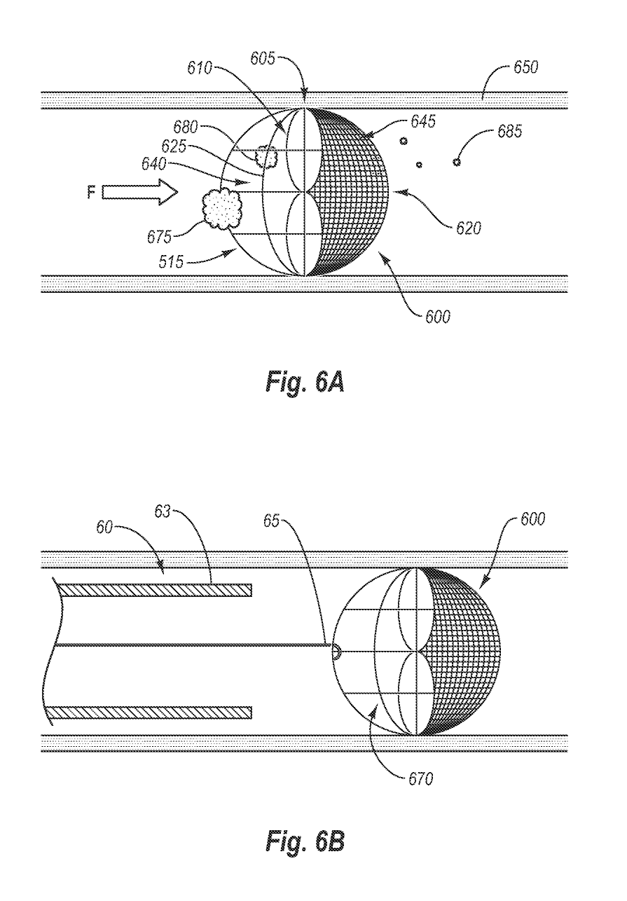

FIG. 6 illustrates a body lumen filter 600 that includes an outer member 605 as well as an inner member 610. The outer member 605 may be similarly configured to the outer member 605 discussed above with body lumen filter 500 (FIG. 5) or may be configured differently. Accordingly, outer structure 605 may include a first hemispherical portion 615, a second hemispherical portion 620, and support 625. Further, the first hemispherical portion 615 may have a first outer filter while the second hemispherical portion 620 may include a second outer filter 635.

The first and second outer filter portions may include first and second filtering gaps 640, 645 defined therein respectively. Further, the body lumen filter 600 may be formed and deployed within a body lumen 650 in a similar manner as the body lumen filter 600.

Further, as illustrated in FIG. 6, the inner member 605 may include one or more rotating blades 655. The rotating blades 655 may rotate about an axis that may be secured to one or more location on the outer member or may be unsecured. The flow of the body fluid within the body lumen 650 may cause the blades 655 to rotate and/or move with respect to the outer member 610. In other examples, the rotating blades 655 may be unsecured to the outer member 610.

In a similar manner as described above with reference to FIGS. 5A-5C, the outer structure 605 may allow some particulates to pass therethrough, such as small embolus 680. As the small embolus 680 enters the outer member 605 and is engaged by the inner member 610. In particular, the small embolus 680 may engage one or more of the fan blades 655, the edges of the fan blades may break up the small embolus 680 into finer emboli 685.

The rotating of the fan blades 655 described above may enhance the efficacy of the inner member 610 in breaking up the small embolus 680 into finer emboli 685. If the finer emboli 685 are sufficiently small as to be less threatening, they may pass through the second outer filter portion. Accordingly, body lumen filters may include active separating structures, including inner portions such as moving inner portions, and rotating fan blades may break up emboli that enter the outer member 605.

FIG. 6B illustrates a process for removing the body lumen filter 600 according to one example. As illustrated in FIG. 6B, a retrieval device 60 may be positioned in proximity to the body lumen filter 600. The retrieval device 60 can include an outer housing 63 and an engagement feature 65, such as a hook. In other embodiments, the retrieval device 60 may include other features to facilitate retrieval of the body lumen filter 600. The engagement feature 65 can be positioned within the outer housing 63 as the retrieval device 60 is moved into proximity with the body lumen filter 600.

Thereafter, the engagement feature 65 can be moved distally out of the outer housing 63. As the engagement feature 65 urged out of the outer housing 63, the engagement feature 65 can come into engagement with the body lumen filter 600 as shown. Thereafter, the engagement feature 65 and the body lumen filter 600 can be drawn into the outer housing 63. Once the body lumen filter 600 is positioned with in the outer housing 63, the retrieval device 60 and body lumen filter 600 can be withdrawn.

Accordingly, apparatuses and methods have been discussed herein for filtering bodily fluid flow (generally described in terms of blood flow) passing through a body lumen (such as a blood vessel). In particular, the apparatuses and methods provided herein include body lumen filters having separating structures operatively associated therewith. According to some examples, the separating structure is a stationary separating structure while in other examples the separating structure is active. Several examples of body lumen filters having both stationary separating structures as well as active separating structures have been generally described above. The separating structures discussed above may be configured to reduce the size of at least some of the emboli that engage the separating structures. For example, the separating structures may break up the emboli until at least a portion of the remaining emboli are below a selected size, such as a size that has been determined to be less threatening.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A body lumen filter, comprising:
a body configured to move between a pre-deployed state and deployed state;
a filtering structure operatively associated with the body to filter particulates; and
a separating structure operatively associated with the body, the separating structure being configured to break up at least one of the particulates.

2. The filter of claim 1, wherein the separating structure is a stationary separating structure.

3. The filter of claim 2, wherein the stationary separating structure includes a plurality of individual members, each of the individual members including at least one feature for engaging particulates, features including at least one of a pointed tip or a cutting edge.

4. The filter of claim 1, wherein the body includes a first portion having a first opening with a first size proximate an exterior of the first portion and a second portion having a second opening with a second size proximate an exterior of the second portion therein, wherein the first opening transitions from the first size to a first smaller size proximate the intermediate portion and wherein the second opening transitions from the second size to a second smaller size proximate the intermediate portion, and wherein the separating structure is located on at least part of an interior side of the second portion.

5. The filter of claim 4, wherein the filtering structure includes a plurality of filtering openings defined in the body.

6. The filter of claim 5, wherein the filtering openings include first filtering openings defined in the first portion and second filtering openings defined in the second portion, the first openings being larger than the second openings.

7. The filter of claim 1, wherein the body includes an inlet having a first size and an outlet having a second size and wherein a wall extends between the inlet and the outlet, wherein the inlet is larger than the outlet and wherein at least a portion of the filtering structure is secured to the wall.

8. The filter of claim 1, wherein the body defines a cavity and further comprising at least one arm extending into the cavity, wherein at least a portion of the separating structure is secured to the arm.

9. The filter of claim 1, wherein the separating structure comprises an active separating structure.

10. The filter of claim 9, wherein the active separating structure includes an outer member having a filtering structure associated therewith an inner member located at least partially within the outer member when the filter is deployed.

11. The filter of claim 10, wherein the outer member is generally spherically shaped and the inner portion is generally spherically shaped.

12. The filter of claim 10, wherein the filtering structure includes a first hemispherical portion and a second hemispherical portion, wherein the filtering structure includes first filtering openings defined in the first hemispherical portion and second filtering openings defined in the second hemispherical portion, the first filtering openings being larger than the second filtering openings.

13. The filter of claim 10, wherein the inner member includes at least one rotating blade.

14. An active filtering device, comprising:
a generally spherically-shaped outer member configured to move between a constricted state and an expanded state, the outer member having a plurality of filtering openings defined therein;
an inner member located at least partially within the outer member when the outer member is in the expanded state; wherein the inner member is configured to move in response to a flow of body fluid to break up at least some of the particulates that enter the outer member.

15. The device of claim 14, wherein the outer member includes a first hemispherical portion and first filtering openings defined therein and a second hemispherical portion having second filtering openings defined therein, wherein the first filtering openings are larger than the second filtering openings.

16. The device of claim 14, wherein the inner member comprises a generally spherically-shaped permeable member.

17. The device of claim 14, wherein the inner member comprises at least one rotating blade.

18. A filtering device, comprising:
a body being configured to move between a constricted state and an expanded state; wherein the body includes a filtering structure including at least first filtering openings defined therein; and
a plurality of individual stationary blending features operatively associated with the body, the plurality of individual stationary blending features being configured to break up particulates carried by a fluid flow.

19. The device of claim 18, wherein the body includes a first conical portion, a second conical portion, and a median portion coupling the first conical portion to the second conical portion and wherein the stationary blending features are located on an interior side of the second conical portion.

20. The device of claim 19, wherein the first conical portion has the first filtering openings defined therein and wherein second filtering openings are defined in the second conical portion.

21. The device of claim 20, wherein the first filtering openings are larger than the second filtering openings.

22. The device of claim 18, wherein the body includes an inlet having first filtering openings defined therein, an outlet having second filtering openings defined therein, and a wall extending between the inlet and the outlet; wherein wall includes a plurality of stationary blending features.

23. The device of claim 22, wherein the first filtering openings are larger than the second filtering openings.

24. The device of claim 19, wherein the body defines a cavity and further comprising at least one arm extending into the cavity, the arm including a plurality of stationary blending features.

25. A method for filtering a body lumen, the method comprising:
providing a body lumen filter comprising:
a body configured to move between a pre-deployed state and deployed state;
a filtering structure operatively associated with the body to filter particulates; and
a separating structure operatively associated with the body, the separating structure being configured to break up at least one of the particulates;
longitudinally elongating the body such that the body lumen filter has a reduced dimension;
delivering the body lumen filter to a desired deployment site within the body lumen; and
longitudinally reducing the body such that the body lumen filter has an enlarged dimension and the at least one anchor applies radial forces to an inner wall of the body lumen.
